(19) United States
(12) Patent Application Publication
(10) Pub. No.: US 2010/0256057 A1
(43) Pub. Date: Oct. 7, 2010

(54) METHOD OF PREVENTING OR TREATING BODY WEIGHT DISORDERS BY EMPLOYING CLUSTERIN

(76) Inventors: Min-Seon Kim, Seoul (KR); Namkoong Churl, Seoul (KR); Pil-Geurn Jang, Gyeonggi-do (KR); Byung-Soo Yoon, Seoul (KR)

Correspondence Address: SHERR & VAUGHN, PLLC, 620 HERNDON PARKWAY, SUITE 320, HERNDON, VA, 20170, US

(21) Appl. No.: 12/599,135

(22) PCT Filed: May 7, 2007
(86) PCT No.: PCT/KR07/02232

§ 371 Date: Nov. 6, 2009

(51) Int. Cl. A61K 38/22; A61K 38/00; A61P 3/04; A61K 31/7088

(52) U.S. Cl. 514/5.3; 514/4.8; 514/44 R; 514/44 A

(57) ABSTRACT

Clusterin possesses an excellent anorexigenic effect and, accordingly, is useful for treating or preventing obesity or an obesity-related disorder. Co-administration of sub-clinical dose of clustrin potentiates the anorexigenic effect of leptin.

### FIELD OF THE INVENTION

The present invention relates to a method of preventing or treating obesity or an obesity-related disorder in a subject, which comprises administering a therapeutically-effective amount of clusterin to the subject; a use of clusterin for the manufacture of a medicament for treating or preventing obesity or an obesity-related disorder in a subject; and a method of treating or preventing anorexia in a lean subject, which comprises administering to the subject a therapeutically-effective amount of a regulator that downregulates clusterin expression.

### BACKGROUND OF THE INVENTION

Obesity occurs as a result of a positive energy balance when the amount of energy intake exceeds the amount of energy expenditure. Under normal conditions, excess energy is stored as fat. It is believed and to some extent known that several factors from the periphery, for example leptin and insulin, are affected by this process (Schwartz M. W. et al., Nature, 404: 661-671 (2000)). These factors can affect the process of food intake (and possibly of energy expenditure) and return the energy balance to neutral. This normal feedback system of controlling energy balance is a protective system that prevents obesity and its related disorders. However, mammals can easily override this feed-back system resulting in obesity-induced diseases and disorders. Obesity-induced disorders are associated with the destabilization of other homeostatic systems, for example glucose or lipid homeostasis, eventually leading to impaired glucose tolerance associated with diminished insulin secretion and increased insulin resistance (Kopelman P. G, Nature, 404: 635-643 (2000)). The exact mechanisms underlying the obesity-induced disorders are not fully understood.

Leptin is a peptide secreted primarily by adipocytes that regulates appetite, energy metabolism and neuronendocrine function (Halaas J. L. et al., Science, 269: 543-546 (1995)). By acting the hypothalamus, leptin diminishes food intake but stimulates energy expenditure, leading to reduction in body fat mass. Deficiency of leptin or leptin receptor causes morbid obesity and hyperphagia in humans and animals (Montague C. T. et al., Nature, 387: 903-908 (1997); and Clement K. et al., Nature, 392: 398-401 (1998)). However, most of human obesity is associated with elevated plasma leptin levels, suggesting that a resistance to leptin is a major cause of obesity in humans and rodents (Maffei M., Nat. Med., 1: 1155-1161 (1995); and Fredrich R. C., Nat. Med., 1: 1311-1314 (1995)). Diminished leptin signaling in the brain or decreased delivery of leptin to central nervous system has been suggested as underlying mechanisms for reduced sensitivity to leptin in obese subjects (Halaas J. L. et al., Proc. Natl. Acad. Sci. USA, 94: 8878-8883 (1997); and Banks W. A., Curr Pharm. Des., 7: 125-133 (2001)). However, the molecular mechanisms for leptin resistance are mostly unknown.

Clusterin (also termed apolipoprotein J) is a 70˜80 kilo-Dalton disulfide-linked heretodimeric protein that is widely distributed in the various tissues and body fluids. Clusterin is encoded by a single gene and the translated product is internally cleaved to produce its α- and β-chains that is linked by five disulfide bonds (Jones S. E. et al., Int. J. Biochem. Cell Biol., 34: 427-431 (2002)). Clusterin is extensively glycosylated such that 30% of the final mass is N-linked carbohydrate.

Clusterin was first described in 1983 as a secreted glycoprotein in ram testis fluid that enhances aggregation (“clustering”) of various cells in vitro (Fritz I. B. et al., Biol. Reprod., 28: 1173-1188 (1983)). Intracellular clusterin is up-regulated not only during various biological processes including differentiation, proliferation and cell death, but also by many different types of stress and cytotoxic insults (Trougakos I. P. et al., Exp. Gerontol., 37: 1175-1187 (2002); and Choi-Miura N. H. et al., Neurobiol. Aging, 17: 717-722 (1996)). Clusterin has been implicated in the lipid transport, cancer cell survival and apoptosis, lipid transport, pancreatic β-cell regeneration and Alzheimer's disease (Jenne D. E. et al., J. Biol. Chem., 266: 11030-11036 (1991); Shannan B. et al., J. Mol. Histol., 37: 183-188 (2006); Kim B. M. et al., Diabetologia, 49: 311-320 (2006); and Matsubara E. et al., Biochem. J., 316: 671-679 (1996)). However, its specific role(s) has not been clearly elucidated. Clusterin is also secreted from the cells and functions as an extracellular chaperon (Humpleys D. et al., J. Biol. Chem., 274: 6875-6881 (1999)).

In an effort to identify plasma protein that modulates leptin actions, clusterin has recently been identified as plasma leptin-binding protein (Bajari T. M., et al., FASEB J., 17: 1505-1507 (2003)). Furthermore, clusterin is highly expressed in the hypothalamus, a regulating center of feeding and energy homeostasis (Danik M., et al., J. Comp. Neurol., 334: 209-227 (1993)).

The present inventors have found that clusterin is an anorexigenic molecule acting in the hypothalamus and decreasing food intake and body weight.

### SUMMARY OF THE INVENTION

Accordingly, it is an object of the present invention to provide a method of treating or preventing body weight disorders in a subject.

It is another object of the present invention to provide an agent for treating or preventing body weight disorders in a subject.

It is a further object of the present invention to provide a pharmaceutical composition for treating or preventing body weight disorders in a subject, comprising said agent.

In accordance with one aspect of the present invention, there is provided a method of treating or preventing obesity or an obesity-related disorder in a subject, which comprises administering a therapeutically-effective amount of clusterin to the subject.

In accordance with another aspect of the present invention, there is provided a use of clusterin for the manufacture of a medicament for treating or preventing obesity or an obesity-related disorder in a subject.

In accordance with a further aspect of the present invention, there is provided a pharmaceutical composition for treating or preventing obesity or an obesity-related disorder in a subject, comprising clusterin and a pharmaceutically acceptable carrier.

In accordance with a still further aspect of the present invention, there is provided a method of treating or preventing anorexia in a lean subject, which comprises administering to the subject a therapeutically-effective amount of a regulator that downregulates clusterin expression.

In the present invention, it is demonstrated for the first time that clusterin induces a significant weight loss in rodents. The effect of clusterin on body weight is comparable to that of leptin, a well-known potent anorexigenic hormone. Clusterin-induced weight reduction is mediated via decreased food intake and increased energy expenditure and oxygen consumption. Consistent with the effect of exogenous administration of clusterin peptide, clusterin gene therapy into, e.g., the hypothalamus and lateral ventricle causes a prolonged decrease in food intake and body weight. It is notable that clusterin gene therapy via, the cerebroventricle is more effective than intra-hypothalamic gene therapy. Thus a delivery of clusterin gene into the cerebroventricular system may be another good therapeutic option in obese subjects.

Clusterin production in the hypothalamus increases following food intake and leptin administration, which indicates that increased hypothalamic clusterin is a physiologic satiety signal. In the animal model of diet-induced obesity, an alteration in hypothalamic clusterin expression according to metabolic change is significantly blunted, suggesting that disordered regulation of clusterin may contribute to hyperphagia and weight gain in obese animals and humans.

As previously reported (van Dijk G. et al., Endocrinology, 146: 547-56 (2005)), central leptin resistance develops in obese mice fed with high fat diet for 7 weeks. Notably, clusterin is able to reduce food intake in these obese animals having leptin resistance. From these facts, one can conclude that central leptin resistance occurs at the physiologic process upstream of clusterin, and clusterin can be used as anti-obesity agent which induces anorexia and weight loss bypassing leptin resistance.

The discovery of leptin raised the hope that a natural compound had been found that could cause weight loss without adverse effects. However, the majority of obese people have high levels of circulating leptin indicating leptin resistance. Thus, the clinical trials of leptin to treat obesity were not so much successful in the most of human obesity (Proietto J. et al., Expert Opin. Investig. Drugs, 12: 373-378 (2003)). In the present invention, co-administration of sub-clinical dose of clusterin significantly enhances leptin sensitivity. Thus clusterin can be used with leptin to enhance the actions of leptin.

Clusterin is highly expressed in the choroid plexus, cerebroventriclular lining epithelium and blood-brain barrier (BBB) (Danik M. et al., J. Comp. Neurol., 334: 209-227 (1993)). It has been shown that clusterin is involved in the transport of amyloid-β through BBB (Zlocovic B. V. et al., Biochem. Biophy. Res. Commun., 205: 1431-1437 (2005)). Therefore, it is possible that clustrein can be used to modulate leptin transport through BBB.

### BRIEF DESCRIPTION OF THE DRAWINGS

The above and other objects and features of the present invention will become apparent from the following description of the invention, when taken in conjunction with the accompanying drawings, which respectively show:

FIG. 1: Graphs showing the anorexigenic effects of clusterin. (a, b) Intracerebroventriclular (ICV) administration of clusterin (1 μg) and leptin (1 μg) following an overnight fast reduced food intake and weight gain in C57BL/6J mice. (n=6˜7). * P<0.01 vs. saline-injected control. (d, e) ICV administration of clusterin (1 μg) blocked Neuropeptide Y (NPY, 2 μg) and Agouti related protein (AGRP, 3 μg)-induced hyperphagia (n=6). * P<0.05 vs. control group. † P<0.05 vs. NPY or AGRP groups. (f) Dose response study of clusterin on feeding effect (n=7). * P<0.05 vs. control. Data are mean±SEM.

FIG. 2: Graphs showing that increased clusterin expression in the hypothalamus and ependimal ling epithelium induces anorexia and fat loss. (a, b) Injection of adenoviruses encoding clusterin (CLU-Ad, 1 μl of 10¹⁰ plaque forming unit) into bilateral mediobasal hypothalamus decreased food intake for 4 days and body weight for 3 days (n=5). Open circles, Adenoviruses encoding Green Fluorescence Protein (GFP-Ad); filled circles, CLU-Ad; * P<0.05. (c)

Epidydimal fat weight was decreased on the 4^{th} day after intra-hypothalamic injection of CLU-Ad (n=5). * P<0.005. (d, e) Increased energy expenditure and oxygen consumption on the 2nd day after intra-hypothalamic injection of CLU-Ad (n=5). ** P<0.01 vs. controls. (f, g, h) Injection of CLU-Ad (5 μl of 10⁸ plaque forming unit) into bilateral lateral ventricle induced a greater decrease in body weight, food intake and epidydimal fat weight, compared to that of intra-hypothalamic injection of CLU-Ad (n=6). Open circles, GFP-Ad; filled circles, CLU-Ad; * P<0.005.

FIG. 3: Graphs showing that decreased clusterin expression in the hypothalamus increases food intake and body weight. (a,b) Bilateral intra-hypothalamic injection of adenoviruses encoding short hairpin RNA against mouse clusterin (CLU-shRNA-Ad, 1 μl of 10¹⁰ plaque forming unit) increased food intake and body weight (n=5). Open circles, GFP-Ad; filled circles, CLU shRNA-Ad; * P<0.05. (c) Increased epidydimal fat weight in CLU-shRNA-Ad injected mice (n=5). ** P<0.05 vs. controls. Data are mean±SEM. (d) Hypothalamic clusterin expression in mice injected with GFP-Ad or CLU-shRNA-Ad.

FIG. 4: Experimental results showing the change of hypothalamic clusterin in normal mice. (a) Hypothalamic clusterin protein expression was increased at 30 min, 1 h and 3 h after regaining of food intake following a 24 h fast (n=5). * P<0.05, ** P<0.01 vs. 24 h fasted group. (b) Effect of ICV leptin (1 μg) on hypothalamic clusterin expression (n=5-6). * P<0.01 vs. saline group.

FIG. 5: Experimental results showing disordered regulation of hypothalamic clusterin expression in the animal model of diet-induced obesity (DIO) and anti-obesity effect of clusterin in DIO mice. (a, b) Food intake and leptin did not increase hypothalamic clusterin expression in mice fed with in high fat diet (HFD, 60% fat) (n=6). (c) ICV administration of clusterin and leptin decreased food intake in mice fed with HFD for 4 weeks. (d) The anorexigenic effect of leptin was attenuated in mice fed with HFD for 7 weeks, suggesting that central leptin resistance develops in these animals. However, anorexigenic effect of clusterin was persistent in mice fed with HFD for 7 weeks. (n=6). * P<0.01 vs. saline group.

FIG. 6: Experimental results showing that clusterin increases leptin sensitivity in terms of food intake and STAT3 activation. (a, b) ICV co-administration of clusterin and leptin at the very low doses that each alone had no significant effect on feeding, induced a significant reduction in 24 h food intake and body weight (n=5-6). * P<0.05, ** P<0.01 vs. saline-injected control. (c) Immunohistochemistry using antibody against phospho-STAT3 (signal transducer activated transcript-3). Leptin increased STAT3 phophorylation in the hypothalamic nuclei such as arcuate nucleus (ARC) and ventromedial hypothalamus (VMH). Clusterin also increased STAT3 phosphorylation in the hypothalamic ARC, chororid plexus, and ventriclular lining ependymal cells. (d) Treatment of clusterin (1 nM) increased expression of phospho-STAT3 and SOCS3 (Suppressor of cytokine signaling) in the primary cultured hypothalamic neurons. (e) Co-treatment of leptin (10 nM) and clusterin (0.01 nM and 0.1 nM), induced STAT3 activation although this small doses of leptin and clusterin did not induce STAT3 activation.

### DETAILED DESCRIPTION OF THE INVENTION

The details of one or more embodiments of the present invention are set forth in the accompanying description below. Other features, objects, and advantages of the present invention will be apparent from the detailed description, drawings, and claims. All publications, patent applications, patents, and other references mentioned herein are incorporated by reference in their entirety.

All technical and scientific terms used herein, unless otherwise defined below, are intended to have the same meaning as commonly understood by one of ordinary skill in the art, and references to techniques employed herein are intended to refer to the techniques as commonly understood in the art, including variations on those techniques or substitutions of equivalent techniques which would be apparent to one of skill in the art. While the following terms are believed to be well understood by one of ordinary skill in the art, the following definitions are set forth to facilitate explanation of the present invention.

The present invention relates to a new agent for treating or preventing body weight disorders, including obesity and anorexia: clusterin.

As used herein, the term “body weight disorders” means disorders which are characterized by an abnormal (e.g., either higher or lower than normal) body weight. For example, individuals having a Body Mass Index (BMI) of less than 18, or more than 25 have an abnormal body weight. Examples of body weight disorders includes obesity, anorexia, cachexia, bulimia, polycystic ovarian disease, hypothalamic syndrome, the Prader-Willi Syndrome, Frohlich's syndrome, GH-deficient subjects, Turner's syndrome and other obesity-related disorders.

As used herein, the term “obesity” means an increase in body weight beyond the limitation of skeletal and physical requirement, as the result of an excessive accumulation of adipose tissue in the body. One non-limiting quantitative definition of “obesity” or “obese”, as used herein is a state in which a subject is at least about 5% over ideal body weight, including but not limited to at least about 10%, 15%, 20%, 30% or more above ideal body weight, wherein at least a portion of the excess body weight is excess adipose tissue. Another useful non-limiting quantitative definition of “obesity” or “obese”, as used herein, is defined as having a body mass index (BMI) of 25 kg/m² or more (National

Institutes of Health, Clinical Guidelines on the Identification, Evaluation, and Treatment of Overweight and Obesity in Adults (1998)). Obesity is associated with and contributes to a variety of different disorders (See, Nishina, P. M. et al., Metab., 43: 554-558(1994); Grundy, S. M. & Barnett, J. P., Dis. Mon., 36: 641-731(1990)).

As used herein, the phrase “obesity-related disorder” refers to any disease, disorder, and/or illness a symptom of which are associated with excess adipose tissue in the subject. The obesity-related disorders are well-known to one of ordinary skill in the art. Non-limiting examples of the obesity-related disorders are hyperphagia, endocrine abnormalities, triglyceride storage disease, heart disease, hypertension, stroke, type II diabetes, impaired glucose tolerance, polycystic ovary syndrome, arthritis, insulin resistance, atherosclerosis, coronary artery disease, hyperlipidemia (e.g., elevated circulating levels of cholesterol, triglycerides and lipoproteins), gallbladder disease, osteoarthritis, sleep apnea, nonalcoholic steatohepatitis, and cancer. It is to be understood that a subject need not necessarily be clinically obese in order to suffer from a disorder associated with obesity. These subjects and the disorders suffered by these subjects that are generally associated with obesity are intended to be included within the scope of the phrase “obesity-related disorders”.

As used herein, “treating” or “treatment” refers to the management and care of a patient for the purpose of combating the disease, condition, or disorder. Treating includes the administration of an active agent to prevent the onset of the symptoms or complications, alleviating the symptoms or complications, or eliminating the disease, condition, or disorder.

As used herein, “preventing” or “prevention” refers to preventing obesity or an obesity-related disorder from occurring if a treating agent is administered prior to the onset of the obese condition. Moreover, if treatment is commenced in subjects already suffering from or having symptoms of obesity or an obesity-related disorder, such treatment is expected to prevent the progression of obesity or the obesity-related disorder.

“Pharmaceutically-acceptable carriers” as used herein include pharmaceutically-acceptable carriers, excipients, or stabilizers which are non-toxic to the cell or mammal being exposed thereto at the dosages and concentrations employed. Often the pharmaceutically-acceptable carrier is an aqueous pH buffered solution. Examples of the pharmaceutically acceptable carriers include buffers such as phosphate, citrate and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counter ions such as sodium; and/or nonionic surfactants such as TWEEN®, polyethylene glycol (PEG), sodium taurodihydrofusidate (STDHF) and PLURONICS®.

The present invention provides for the first time a correlation between clusterin and a reduction in the prevalence of obesity and obesity-related disorders in subjects at risk for obesity.

Accordingly, a method of treating or preventing obesity in a subject is provided. In some embodiments, the method comprises administering clusterin to the subject, whereby obesity in the subject is treated or prevented. Additionally, a method of treating or preventing an obesity-related disorder in a subject in need of such treatment is provided. In some embodiments, the method comprises administering clusterin to the subject, whereby the obesity-related disorder in the subject is treated or prevented. In the above embodiments, clusterin may be administered as pharmaceutically acceptable salts. Such pharmaceutically acceptable salts include the gluconate, lactate, acetate, tartarate, citrate, phosphate, maleate, borate, nitrate, sulfate, and hydrochloride salts.

In some embodiments, the obesity-related disorder is selected from the group consisting of hyperphagia, endocrine abnormalities, triglyceride storage disease, heart disease, hypertension, stroke, type II diabetes, impaired glucose tolerance, polycystic ovary syndrome, arthritis, insulin resistance, atherosclerosis, coronary artery disease, hyperlipidemia, gallbladder disease, osteoarthritis, sleep apnea, nonalcoholic steatohepatitis, and cancer.

The invention further provides a method for treating obesity or obesity-related disorders in a mammal by administering clusterin in combination with one or more further active agents in any suitable ratios. When used in combination with one or more further active agents, the combination of compounds is preferably a synergistic combination. Synergy occurs when the effect of the compounds when administered in combination is greater than the additive effect of the compounds when administered as a single agent. In general, a synergistic effect is most clearly demonstrated at sub-optimal concentrations of the compounds. Such further active agents may be selected from antiobesity agents, antidiabetic agents, antihyperlipidemic agents, antihypertensive agents and agents for the treatment of complications resulting from or associated with obesity. Preferably, such further active agent is leptin. In some embodiments, clusterin and leptin may be administered at a weight ratio ranging from 0.01:1 to 1:0.01, preferably, from 0.05:1 to 1:0.05.

With reference to the therapeutic methods, a subject can be any subject in need of preventing or treating obesity and/or related disorders. For example, a subject that is already obese can be treated in order to reduce the subject's body weight by reducing excess adipose tissue, or even maintain a subject's body weight and prevent further significant adipose tissue deposition in the subject. For subjects treated to prevent obesity or disorders associated obesity, the subject can be considered in need of such a treatment if, for example, the subject was predisposed to obesity or disorders associated with obesity. A subject can be considered predisposed for obesity or disorders associated with obesity if, for example, the subject had a genetic predisposition for obesity, or had at one time suffered from obesity and was at risk of becoming obese again.

A subject can be any vertebrate species. The methods of the present invention are particularly useful in the treatment of warm-blooded vertebrates. Thus, the presently claimed subject matter concerns mammals. More particularly, provided is the treatment of mammals such as primates, including humans, as well as those mammals of importance due to being endangered (such as Siberian tigers), of economic importance (animals raised on farms for consumption by humans) and/or social importance to humans (animals kept as pets or in zoos), for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of disease in livestock, including, but not limited to domesticated swine (pigs and hogs), ruminants, horses, poultry, and the like.

The present invention also provides a pharmaceutical composition comprising clusterin and a pharmaceutically acceptable carrier for preventing or treating obesity or disorders associated with obesity. The content of clusterin in the pharmaceutical composition may ranges from 0.1 to 99.5% by weight, more preferably, 0.5 to 90% by weight. Additional formulation and dose preparation techniques have been described in the art (See, those described in U.S. Pat. No. 5,326,902; U.S. Pat. No. 5,234,933; and PCT International Publication No. WO 93/25521).

For therapeutic applications, a therapeutically effective amount of the inventive pharmaceutical composition is administered to a subject. A “therapeutically effective amount” or “effective amount” is an amount of the therapeutic composition sufficient to produce a measurable biological response, such as but not limited to a reduction in body weight or food intake and increase of energy expenditure.

Further, the pharmaceutical composition can be administered alone, or in combination with other therapies (e.g., diet regimens and exercise) and/or therapeutics.

For instance, the pharmaceutical composition may comprise clusterin in combination with one or more further active agents in any suitable ratios. Such further active agents may be selected from antiobesity agents, antidiabetic agents, antihyperlipidemic agents, antihypertensive agents and agents for the treatment of complications resulting from or associated with obesity. Preferably, such further active agent is leptin, and the pharmaceutical composition may comprise clusterin and leptin at a weight ratio ranging from 0.01:1 to 1:0.01, preferably, from 0.05:1 to 1:0.05.

For the purposes described above, clusterin or a pharmaceutical composition comprising the same can normally be administered systemically or topically, usually by intranasal or parenteral administration. The route of administration may include but is not limited to intra-hypothalamic, intracerebroventricular, intrathecal, oral, intraoral, rectal, transdermal, buccal, pulmonary, subcutaneous, intramuscular, intradermal, intracolonic, intraoccular, intravenous, or intestinal administration. The pharmaceutical composition is formulated according to the route of administration based on acceptable pharmacy practice (Fingi et al., in The Pharmacological Basis of Therapeutics, Ch. 1, p.1, 1975; Remington's Pharmaceutical Sciences, 18th ed., Mack Publishing Co, Easton, Pa., 1990). Thus, the formulations may be in the form of a tablet, pill, powder, sachet, elixir, suspension, emulsion, solution, syrup, aerosol, soft and hard gelatin capsules, sterile injectable solution, sterile packaged powder and the like. The compositions of the present invention may be formulated so as to provide quick, sustained or delayed release of the active ingredient after administration to a patient by employing any of the procedures well known in the art.

Preferably, the composition of the present invention can be administered into the cerebrospinal fluid (CSF) in an intranasal form (e.g., intranasal spray) via topical use of suitable intranasal vehicles, or via transdermal routes, using transdermal skin patches. When administered in the form of a transdermal delivery system, the dosage administration will, of course, be continuous rather than intermittent throughout the dosage regimen.

Various compositions and forms of administration are provided and are generally known in the art. Other compositions for administration include liquids for external use, and endermic liniments (ointment, etc.), suppositories, and pessaries that comprise one or more of the active substance(s) and can be prepared by known methods.

The pharmaceutical composition of the present invention is typically administered parenterally in dosage unit formulations containing standard, well-known nontoxic physiologically acceptable carriers, adjuvants, and vehicles as desired. The term “parenteral” as used herein includes, but is not limited to intravenous, intramuscular, intra-arterial injection, or infusion techniques. Injectable preparations, e.g., sterile injectable aqueous or oleaginous suspensions, are formulated according to the methods known in the art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable carriers and solvents that can be employed are water, Ringer's solution, and isotonic sodium chloride solution. In addition, sterile, fixed oils are conventionally employed as solvents or suspending media. For this purpose, any bland fixed oil can be employed including synthetic mono- or diglycerides. In addition, fatty acids such as oleic acid may be used in the preparation of injectable solution or suspension.

Representative, non-limiting carriers include neutral saline solutions buffered with phosphate, lactate, Tris, and the like. Of course, one purifies the carrier sufficiently to render it essentially free of undesirable contaminants such that it does not cause any untoward reactions in the individual receiving the carrier and therapeutic composition(s).

Actual dosage levels of clusterin in a pharmaceutical composition can be varied so as to administer an amount of the active compound(s) that is effective to achieve the desired therapeutic response for a particular subject. The selected dosage level will depend upon a variety of factors including, but not limited to the activity of the pharmaceutical composition, the formulation, the route of administration, combinations with other drugs or treatments, and the physical condition and prior medical history of the subject being treated. In some embodiments, a minimal dose is administered, and the dose is escalated in the absence of dose-limiting toxicity. The determination and adjustment of a therapeutically effective dose, as well as evaluation of when and how to make such adjustments, are well known to those of ordinary skill in the art of medicine.

By way of general guidance, the daily oral dosage of the active ingredient, when used for the indicated effects, will range from about 0.001 to 1,000 mg/kg of body weight, preferably, from about 0.01 to 100 mg/kg of body weight per day.

Intravenously, the daily dosage of the active ingredient may range from 0.001 ng to 100.0 ng per min per Kg of body weight during a constant rate infusion. Such constant intravenous infusion can be preferably administered at a rate of 0.01 ng to 50 ng per min per Kg body weight and, most preferably, at 0.1 ng to 10.0 ng per min per Kg body weight. The composition of this invention may be administered in a single daily dose, or the total daily dosage may be administered in divided doses of two, three, or four times daily. The composition of this invention may also be administered by a depot formulation that will allow sustained release of the drug over a period of days/weeks/months as desired.

One aspect of the present invention relates to a method of treating obesity or an obesity-related disorder in a subject comprising delivering a viral vector comprising a gene encoding clusterin to the subject.

The heterologous clusterin gene may be delivered to the subject using a vector or other delivery vehicle. DNA delivery vehicles can include viral vectors such as adenoviruses, adeno-associated viruses, helper dependent adenoviruses, and retroviral vectors. See, for example: Chu et al., Gene Ther., 1: 292-299 (1994); Couture et al., Hum. Gene Ther., 5: 667-277 (1994); and Eiverhand et al., Gene Ther., 2: 336-343 (1995). Non-viral vectors which are also suitable include DNA-lipid complexes, for example, liposome-mediated or ligand/poly-L-Lysine conjugates, such as asialoglyco-protein-mediated delivery systems. See for example: Felgner et al., J. Biol. Chem. 269: 2550-2561 (1994); Derossi et al., Reston: Neurol. Neuros., 8: 7-10 (1995); and Abcallah et al., Biol. Cell 85: 1-7(1995).

If a viral vector is chosen as the delivery vehicle, it may be one which is capable of integrating into the host genome, so that the gene can be expressed permanently, but this is not required. In cases where the vector does not integrate into the host genome, the expression of the gene may be transient rather than permanent.

The vector may be administered to the host, generally by intra-hypothalamic, intracerebroventricular or intrathecal injection, but may also be intravenous, intramuscular, intraperitoneal, oral, subcutaneous or other form of delivery. Suitable titers will depend on a number of factors, such as the particular vector chosen, the host, strength of promoter used and the severity of the disease being treated. For mice, an adenovirus vector is preferably administered as an injection at a dose range of from about 1.0×10⁶ to about 1.0×10¹⁰ plaque forming units (PFU) per gram body weight. Higher amounts are also useful, and up to 10¹² particles may be used.

If the effect desired is a permanent one rather than a transient one, it is preferred that a helper dependent viral vector be utilized. Promoters that are suitable for use with these vectors include the Moloney retroviral LTR, CMV promoter and the mouse albumin promoter. Replication competent free virus can be produced and injected directly into the animal or humans or by transduction of an autologous cell ex vivo, followed by injection in vivo as described in Zatloukal et al., Proc. Natl. Acad Sci. USA, 91: 5148-5152 (1994).

The clusterin coding sequence can also be inserted into plasmid for expression of clusterin in vivo or ex vivo. For in vivo therapy, the coding sequence can be delivered by direct injection into tissue or by intravenous infusion. Promoters suitable for use in this manner include endogenous and heterologous promoters such as CMV. The coding sequence can be injected in a formulation comprising a buffer that can stabilize the coding sequence and facilitate transduction thereof into cells and/or provide targeting, as described in Zhu et al., Science, 261: 209-211 (1993).

Expression of the clusterin coding sequence in vivo upon delivery for gene therapy purposes by either viral or non-viral vectors can be regulated for maximal efficacy and safety by use of regulated gene expression promoters as described in Gossen et al., Proc. Natl. Acad. Sci. USA, 89: 5547-5551(1992). For example, the clusterin coding sequence can be regulated by tetracycline responsive promoters. These promoters can be regulated in a positive or negative fashion by treatment with the regulator molecule.

For non-viral delivery of the clusterin coding sequence, the sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, as described in Wu and Wu, J. Biol. Chem., 262: 4429-4432 (1987); insulin, as described in Hucked et al, Biochem. Pharmacol., 40: 253-263 (1990); galactose, as described in Plank et al., Bioconjugate Chem., 3: 533-539 (1992); lactose, as described in Midoux et al., Nucleic Acids Res., 21: 871-878 (1993); or transferrin, as described in Wagner et al., Proc. Natl. Acad. Sci. USA, 87: 3410-3414 (1990). Other delivery systems include the use of liposomes to encapsulate DNA comprising the clusterin gene under the control of a variety of tissue-specific or ubiquitously-active promoters, as described in Nabel et al., Proc. Natl. Acad. Sci. USA, 90: 11307-11311 (1993), and Philip et al., Mol. Cell Biol., 14: 2411-2418 (1994). Further non-viral delivery suitable for use includes mechanical delivery systems such as the biolistic approach, as described in Woffendin et al., Proc. Natl. Acad. Sci. USA, 91: 11581-11585 (1994). Moreover, the clusterin coding sequence can be delivered through deposition of photopolymerized hydrogel materials. Other conventional methods for gene delivery that can be used for delivery of the clusterin coding sequence include, for example, use of hand held gene transfer particle gun, as described in U.S. Pat. No. 5,149,655; use of ionizing radiation for activating transferred gene, as described in U.S. Pat. No. 5,206,152 and PCT publication No. WO 92/11033.

The present invention further provides a method of treating or preventing anorexia in a lean subject, which comprises administering to the subject a therapeutically-effective amount of a regulator that effectively downregulates clusterin expression.

The anorexia may be a cancer-related anorexia, anorexia nervosa, false anorexia, etc.

Representative examples of the regulator include antisense RNAs, interfering RNAs, short hairpin RNAs, and small interfering RNAs capable of mediating RNA interference against clusterin gene expression, and expression vectors thereof, which can be used for downregulating clusterin expression at its mRNA level; transcription inhibitors of clusterin; translation inhibitors of transcribed clusterin mRNA; and inhibitors of clusterin localization. Among them, a small interfering RNA or short hairpin RNA and its expression vector are preferable because they can specifically and potently downregulate clusterin gene expression even when a small amount is applied. More preferably, the regulator is a short hairpin RNA and its expression vector, a cDNA of said short hairpin RNA having the nucleotide sequence of SEQ ID NO: 1.

The small interfering RNA or short hairpin RNA may be delivered to the subject using a viral or non-viral vector or other delivery vehicle, as described in the above for the delivery of a heterologous clusterin gene to the subject.

Further, the regulators of clusterin expression may be formulated to pharmaceutical compositions or preparations and administered to a subject via various routes, as described above with regard to clusterin.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### Reference Example

### <Animals>

Adult male C57BL/6J mice and Spraque-Dawley rats were obtained from Daehan Laboratory Animal Research (Seoul, Korea) and Lep^{-/-} mice from Japan SLC Inc. (Shizuoka Ken, Japan), respectively. Mice were fed standard chow (Samyang Co, Seoul, Korea) ad libitum unless specifically mentioned. They were housed under controlled temperature (24° C.) and a 12 hr light-dark cycle, with light from 06:00 a.m. to 06:00 p.m. All procedures were approved by the Institutional Animal Care and Use Committee of the Asan Institute for Life Sciences (Korea).

### <Statistical Analysis>

Data are presented as mean±SEM. Statistical analysis was performed using SPSS-PC 12 (Chicago, Ill.). Statistical significance among the groups was tested with one-way analysis of variance (ANOVA) followed by a post hoc LSD test, or an unpaired Student's t test when appropriate. Repeated ANOVA was used for studies of CLU-Ad or CLU-shRNA experiments. Two-way ANOVA was used for the study of ICV leptin in CLU-RNAi-Ad-expressing mice. Significance was defined as P<0.05.

### Example 1

### Clusterin Caused Anorexia and Weight Loss

Clusterin has two isoforms, long secretory (70-80 kilo Dalton) and short nuclear clusterin (˜45 kilo Dalton). Recombinant human secretory clusterin (Adipogen, Seoul, Korea), which is disulfide-linked and glycosylated, like human plasma clusterin, was used in this experiment. To investigate the effect of clusterin on food intake and body weight, 26 gauge-permanent cannulae (Plastics One Inc., Roanoke, Va.) was cannulated into the 3^{rd} ventricle (coordination of cannulation: 1.8 mm caudal to bregma and 5.0 mm ventral to the sagittal sinus) of C57BL/6J mice (n=6 for each experimental group) using streotaxic surgery. After 1 week-recovery period, saline, clusterin or leptin was injected to the mice via permanent cannulae following an overnight fast. Peptides were dissolved in 0.9% saline and administered intra-cerebroventricularly (ICV) in 2 μl of volume.

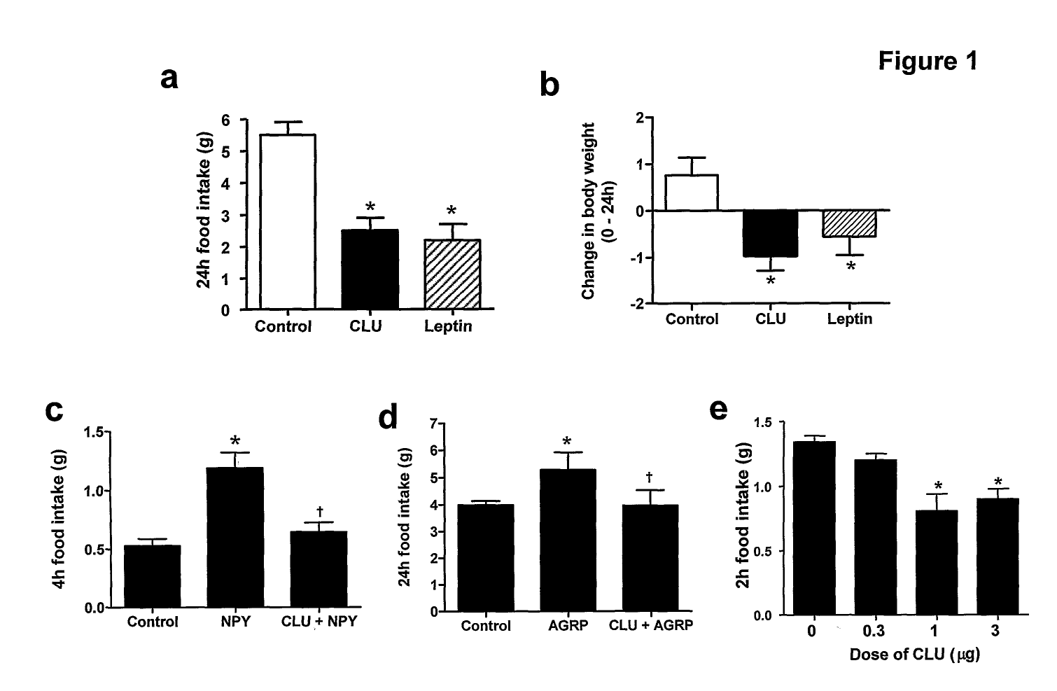

As shown in FIG. 1a, ICV administration of secretory clusterin (1 μg) reduced fast-induced feeding by 60% in lean C57BL/6J mice. The anorexigenic effect of clusterin was as potent as the same amount of leptin (R&D systems, Minneapolis, Minn.). Both clusterin and leptin significantly decreased body weight gain for 24 h post-injection (FIGS. 1, b). Clusterin (1 μg) also blocked hyperphagia induced by administration of neuropeptide Y (NPY, 2 μg) and Agouti related protein (AGRP, 3 μg) when co-administered with ICV (FIGS. 1, c and d). Dose-response study revealed that clusterin at the dose of 1 μg induced a greatest decrease in food intake (FIGS. 1, e). Higher dose of clusterin did not produce a further reduction in food intake. Repeated ICV injection of clusterin (1 μg/day) reduced food intake and prevented weight gain after the 3^{rd} treatment day.

### Example 2

### Clusterin Gene Therapy Decreased Food Intake and Body Weight

To further investigate the effect of clusterin gene therapy on food intake and body weight, we generated adenoviruses encoding full length secretory clusterin. The cDNA encoding full-length rat clusterin (from DR. Bon-Hong Min, Korea University; GenBank Accession No.: BC061534) was inserted in the EcoRI/XhoI sites of the pAdTrack-CMV shuttle vector (Stratagene, La Jolla, Calif.). The resulting vector was then electroporated into BJ5138 cells (from DR. In-Kyu Lee, Kyongbuk National University, Korea) containing the Adeasy adenoviral vector to produce the recombinant adenoviral plasmid. The recombinants were amplified in HEK-293 cells and isolated and purified using CsCl (Sigma) gradient centrifugation. The preparations were collected and desalted, and the titers were determined using Adeno-X Rapid titer (BD Bioscience, San Jose, Calif.) according to the manufacturer's instructions.

To increase hypothalamic clusterin expression, 1×10¹⁰ p.f.u. of adenoviruses expressing clusterin (CLU-Ad) was micro-injected into bilateral mediobasal hypothalamus (iMBH) (1 μl each side, coordination of injection: 1.8 mm caudal to bregma, 5.8 mm ventral and 0.2 mm lateral to the sagittal sinus) of

C57BL/6J mice (n=5) using infusion pump (Harvard Apparatus) at a rate of 1 μl/5 min/each side. 1×10¹⁰ p.f.u. of adenoviruses expressing green fluorescent protein (GFP-Ad) as a control was also injected to C57BL/6J mice (n=5) into bilateral mediobasal hypothalamus. A proper injection of adenoviruses was confirmed by examining GFP expression in the hypothalamic area of the mice at the end of the study. Food intake and body weight were daily monitored at early light phase (9-10 A.M.) for 4 days. Mice were sacrificed at the 4^{th} day of adenovorus injection and epidydimal fat pad was collected and weighted.

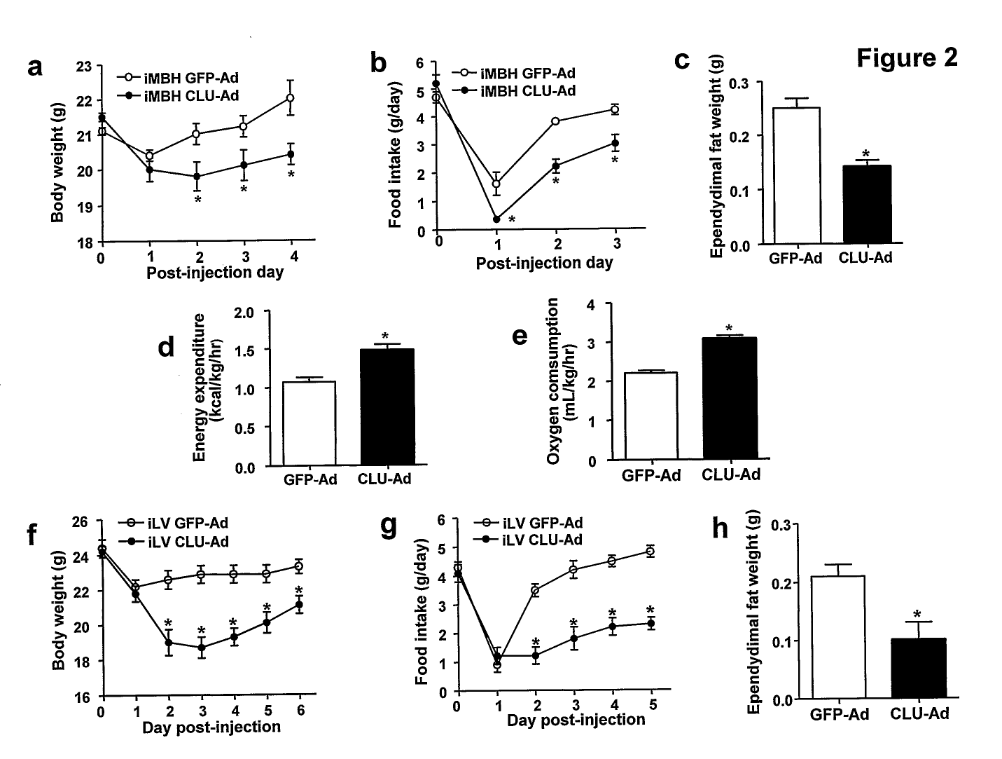

Clusterin gene therapy into the hypothalamus caused a significant decrease in body weight (FIGS. 2, a) and abdominal fat weight at day 4 after the injection (FIGS. 2, c). In mice received CLU-Ad, food intake decreased for 3 days (FIGS. 2, b). On the 2^{nd} day of adenovirus injection, we measured O₂ consumption (VO₂), energy expenditure (EE) and respiratory quotient (RQ) using an Oxymax apparatus (Columbus Instruments, Columbus, Ohio). As shown in FIGS. 2, d and e, oxygen and energy consumption increased in mice injected with CLU-Ad compared to controls with GFP-Ad. These changes in energy metabolism would result in decreased body fat mass in those mice.

Meanwhile, GFP-Ad or CLU-Ad (2.5×10⁸ p.f.u) was also administered into the bilateral lateral ventricle (iLV) of C57BL/6J mice (n=6) (2.5 μl each side, coordination of injection: 0.28 mm caudal to bregma, 2 mm ventral and 1 mm lateral to the sagittal sinus), and changes in food intake, body weight and abdominal fat weight were determined as described above. As shown in FIGS. 2, f, g and h, clusterin gene therapy into the cerebroventricle caused greater decreases in food intake and body weight compared with the clusterin gene therapy into the hypothalamus, even though only one-fortyth of the dose of adenoviruses used for the hypothalamus administration was employed.

### Example 3

### Clusterin RNAi Gene Therapy Increased Food Intake and Body Weight

In order to examine the effect of decreased clusterin expression in the hypothalamus, an adenovirus expressing short hairpin (small inhibitory) RNA that inhibits mouse clusterin expression (CLU-shRNA-Ad) was generated using the same method as described in Example 2, except for replacing clusterin cDNA with a cDNA for a short hairpin RNA (shRNA) against mouse clusterin. A short hairpin RNA is a sequence of RNA that makes a tight hairpin turn that can be used to silence gene expression via RNA interference (McIntyre G, et al., BMC Biotechnol. 6: 1 (2006)). The cDNA for mouse clusterin shRNA had the nucleotide sequence of 5′-CATAGAACTTCATGCAGGTAT(antisense)

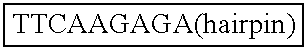

ATACCTGCATGAAGTTCTA(sense)TTTTTTCCAA-3′ (SEQ ID NO: 1) and was designed targeting nucleotides 444˜462 of murine clusterin sequence (GenBank Accession No.: NM_{—}013492).

It was confirmed that treatment of CLU-shRNA-Ad knocked down clusterin expression in TM3 mouse Leydig cells (from DR. Chan-Soo Shin, Seoul National University) before in vivo study.

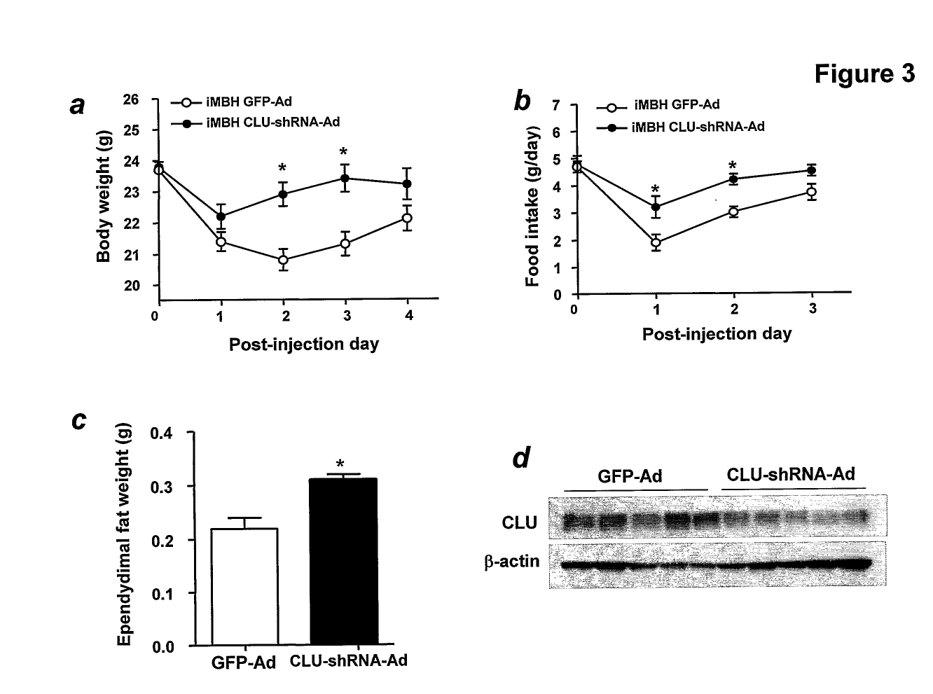

Injection of CLU-shRNA-Ad (1×10¹⁰ p.f.u. each side) in the bilateral mediobasal hypothalamus of C57BL/6J mice (n=10) was performed using the same method described in Example 2. Injection of CLU-shRNA-Ad significantly reduced food intake, body weight and epidydimal fat weight (FIGS. 3; a, b and c).

To confirm whether injection of CLU-shRNA-Ad can reduce hypothalamic clusterin expression, some of mice injected with adenoviruses were sacrificed at the 2nd day after adenovirus injection. The hypothalami were collected as previously described (Namkoong C., et al. Diabetes, 54:63-8 (2005)) to determined hypothalamic clusterin expression and stored in −80° C. till assayed. The hypothalamus was lysed by 200 μl extraction buffer (20 mM Tris-Hcl (pH 7.4), 1 mM EDTA, 140 mM NaCl, 1% NP40, 1 mM Na₃VO₄, 1 mM PMSF, 50 mM NaF, 10 μg/ml Aprotinin) to obtain hypothalamic lysates. Each of the hypothalamic lysates (30 μg protein) was subjected to immunoblotting with an antibody against clusterin β-chain (Santa Creutz) in order to determine clusterin expression. As shown in FIG. 3 d, intrahypothalamic injection of CLU-shRNA-Ad significantly decreased hypothalamic clusterin expression.

Thus, it was demonstrated that inhibition of clusterin expression can increase food intake and body weight.

### Example 4

### Hypothalamic Clusterin is Regulated by Metabolic Signals

If clusterin plays a role in the hypothalamic regulation of energy homeostasis, hypothalamic clusterin expression may be affected by feeding state. Thus hypothalamic clusterin expression in altered metabolic state was examined as follows.

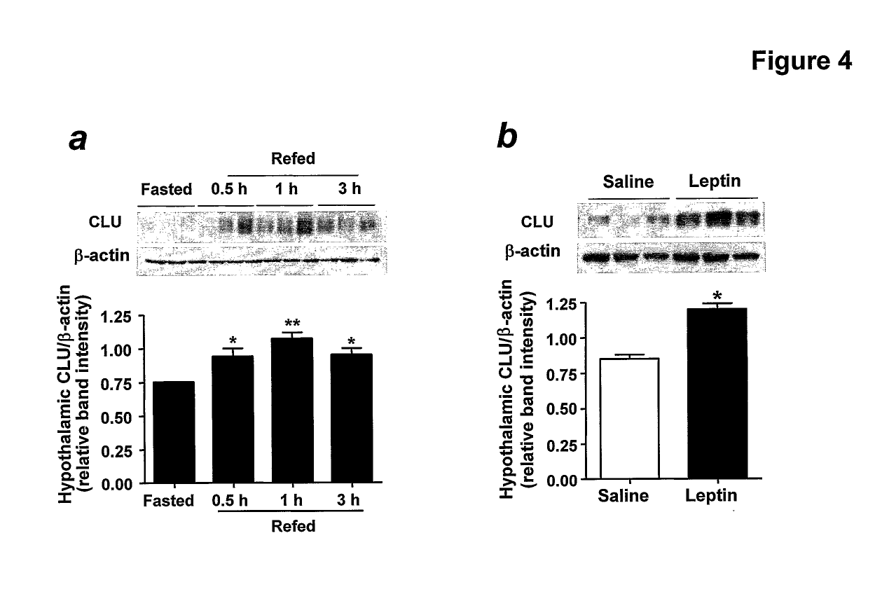

To investigate the change in hypothalamic clusterin expression according to feeding state, the hypothalami were collected from Sprague-Dawley rats in 24 hour fasted state (n=5), 1 hour refed state following 24 hour fast (n=5), and 3 hour refed state following 24 hour fast (n=5). Hypothalamic clusterin expression was determined using clusterin immunoblotting as described in Example 3. As shown in FIG. 4 a, regaining of feeding following 24 hour fasting increased clusterin levels in the hypothalamus.

As plasma levels of leptin, a potent anorexigenic hormone, are increased by food intake and, the effect of leptin on hypothalamic clusterin expression was also investigated. One hour following ICV administration of leptin (1 μg), hypothalami of C57BL/6J mice (n=5) were collected and subjected to clusterin immmunoblotting as above. As shown in FIG. 4b, leptin treatment significantly increased clusterin expression in the hypothalamus.

### Example 5

### The Change and Effect of Clusterin in Obese Animals

To investigate hypothalamic clusterin expression in animal model of diet induced obesity (DIO), C57BL/6J mice (n=6 for each experimental group) were fed with either high fat diet (HFD, fat 60%, Research Diet, Inc., New Brunswick, N.J.) or low fat diet (LFD, 10% fat) for 8 weeks.

The hypothalami were collected at the end of HFD treatment in 24 hour fasted and 1 hour refed state and determined hypothalamic clusterin expression using clusterin immunoblotting as described in Example 4.

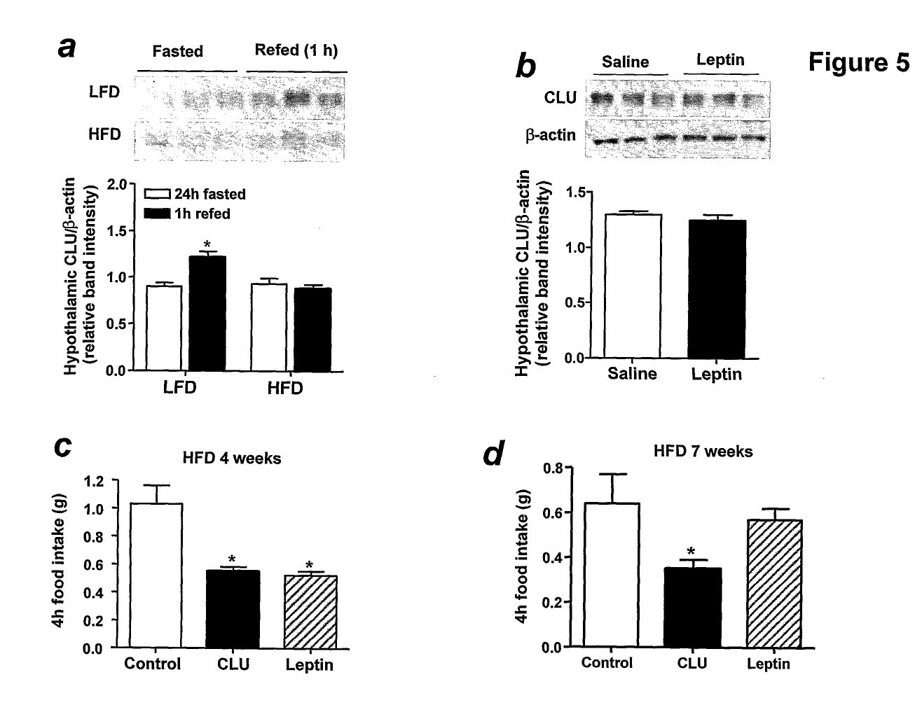

In fasted state, hypothalamic clusterin expression in HFD-fed obese mice was not significantly different from that in LFD-fed mice (FIG. 5, a). However, feeding-induced change in hypothalamic clusterin was significantly blunted in HFD-fed mice (FIGS. 5, a). Similarly, leptin did not induced change in hypothalamic clusterin expression in HFD-fed mice (FIGS. 5, b). Failure to increase hypothalamic clusterin expression by these factors may hamper to produce satiety in obese mice that may contribute to development of obesity.

To investigate the effects of leptin and clusterin in DIO mice, we injected saline, leptin (1 μg) and clusterin (1 μg) ICV to the mice (n=6 for each experimental group) at 2, 4, 6 and 7 weeks during HFD treatment. ICV administration of leptin and clusterin induced anorexia in mice fed with HFD for initial 4 week period of HFD (FIGS. 5, c). However, the anorexigenic effect of leptin became insignificant in mice with 7 weeks-HFD treatment, suggesting that DIO mice had central leptin resistance. In contrast, ICV administration of clusterin caused suppression of feeding even in mice fed with HFD for 7 weeks in these mice (FIGS. 5, d).

### Example 6

### Clusterin Increases Leptin Sensitivity

To investigate the interaction of leptin and clusterin in feeding regulation, leptin and clusterin, alone or together, were administered ICV to C57BL/6J mice (n=5 for each experimental group) following 24 hour fast. 0.1 μg each of leptin and clusterin, that alone did not induce anorexia and weight loss, were administered to the mice.

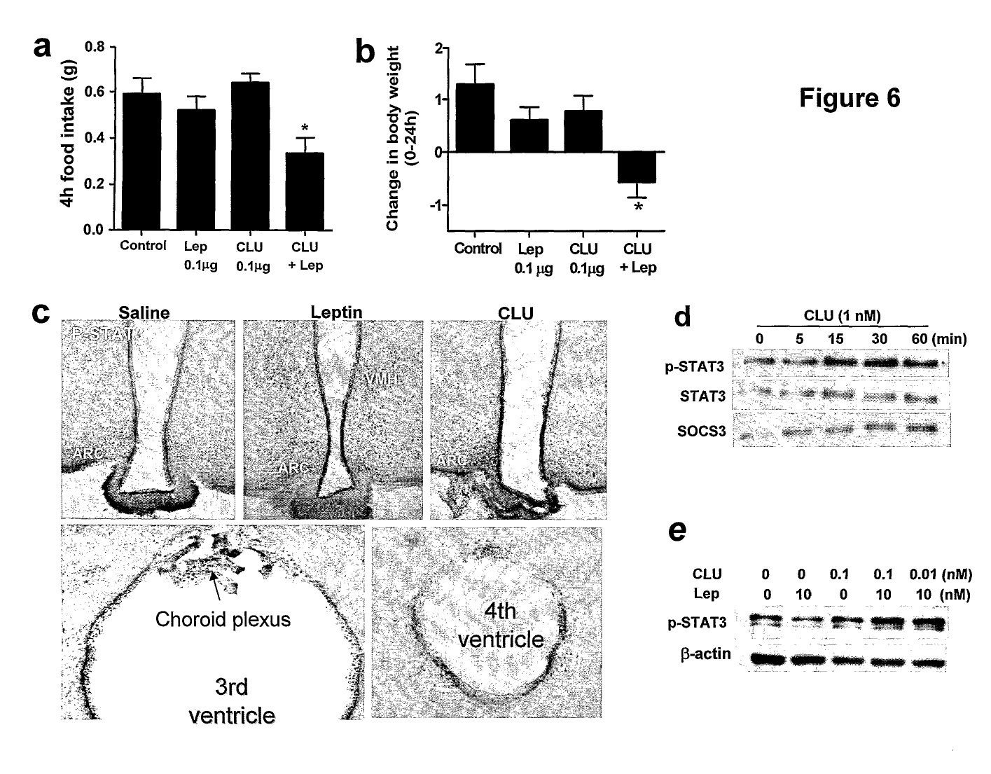

As shown in FIGS. 6a and b, co-administration of the sub-effective dose of clusterin and leptin did induce a significant reduction in food intake and body weight gain (FIGS. 6, a and b). This data suggests that clusterin therapy can increase leptin sensitivity when administered with leptin.

Next, the signaling pathway by which leptin and clusterin act to regulate food intake was examined. The previous study have shown that leptin increases expression of phosphorylated STAT3, a marker for STAT3 activation, in several hypothalamic nuclei (Hou L. et al., Endocrinology, 145: 2516-2523(2004)). To compare the signaling pathway of leptin and clustrein, mice were injected ICV with leptin (1 μg) and clusterin (1 μg). At 30 min after ICV injection, mice were perfused with 10% formalin for 15 min using a peristaltic pump through heart under anesthesia. To verify STAT3 phosphorylation in the hypothalamus, immunohistochemistry using antibody against phospho-Y⁷⁰⁵-STAT3 (Cell Signaling, Beverly, Mass.) was performed as previously described (Hou L. et al., Endocrinology, 145: 2516-2523 (2004)).

Consistent with the previous report, leptin increased expression of phospho-STAT3 in the multiple hypothalamic nuclei including the arcuate nucleus (ARC) and ventromedial nucleus (VMH) (FIG. 6c). Clusterin also induced STAT3 activation in the ARC (FIG. 6c), retrochiasmatic and premammilary nuclei, but not in VMH, dorsomedial nucleus and paraventricular nucleus.

The effect of clusterin in hypothalamic neurons was further confirmed as follows. The fetal rat hypothalami were collected at embryonic day 19-20 and primarily cultured as previously described (Canick J. A., et al. Brain Res., 372: 277-282 (1986)). Differentiation was induced by culturing cells in neurobasal medium (Invitrogen, Carlsbad, Calif.) supplemented with 5 μg/ml insulin, 100 μ/ml iron-free human transferrin, 100 μM putrescin, 30 nM sodium selenite, and 20 nM progesterone for 7-8 days. Cells were treated with clusterin and/or leptin for indicated time following 2 hour-serum starvation.

In cultured hypothalamic neurons, clusterin (10 nM) increased expressions of phopho-STAT3 and suppressor of cytokine signaling (SOCS)-3 (FIGS. 6d) at 15, 30 and 60 min of treatment. In consistent with in vivo findings (FIGS. 6a and b), co-treatment of clusterin (0.01 and 0.1 nM) and leptin (10 nM) at the lower doses that each alone can not induce STAT3 activation, induced a significant further activation of STAT3 (FIG. 6e). These data indicate that clusterin augments leptin-signaling pathway.

While the invention has been described with respect to the above specific embodiments, it should be recognized that various modifications and changes may be made to the invention by those skilled in the art which also fall within the scope of the invention as defined by the appended claims.

1-44. (canceled)

45. A method comprising:
at least one of preventing and treating at least one of obesity and an obesity-related disorder in a subject, comprising administering a therapeutically-effective amount of at least one of clusterin and a clusterin activator to the subject.

46. The method of claim 45, wherein said obesity-related disorder comprises at least one of hyperphagia, endocrine abnormalities, triglyceride storage disease, heart disease, hypertension, stroke, type II diabetes, impaired glucose tolerance, polycystic ovary syndrome, arthritis, insulin resistance, atherosclerosis, coronary artery disease, hyperlipidemia, gallbladder disease, osteoarthritis, sleep apnea, nonalcoholic steatohepatitis and cancer.

47. The method of claim 45, wherein the subject is a mammal.

48. The method of claim 47, wherein the mammal comprises at least one of a rodent, a swine, a ruminant and a primate.

49. The method of claim 48, wherein the primate comprises a human.

50. The method of claim 45, wherein the clusterin comprises a form including a pharmaceutical preparation.

51. The method of claim 50, wherein said pharmaceutical preparation comprises at least one of a tablet, capsule, injectable solution, injectable suspension, intranasal spray and intradermal patch.

52. The method of claim 45, wherein the clusterin is administered at a daily dosage between approximately 0.001 mg/kg of body weight and 1000 mg/kg of body weight.

53. The method of claim 45, wherein the clusterin is administered at least one of intra-hypothalamically, intracerebroventricularly, intravenously, intraperitoneally, orally, transdermally and intranasally.

54. The method of claim 45, wherein said clusterin activator comprises leptin.

55. The method of claim 54, wherein the clusterin is administered in combination with the leptin.

56. The method of claim 55, wherein the clusterin and the leptin are administered at a weight ratio ranging between approximately 0.01:1 and 1:0.01.

57. A method comprising:
at least one of preventing and treating at least one of obesity and an obesity-related disorder in a subject, comprising administering a therapeutically-effective amount of a gene encoding clusterin to the subject.

58. The method of claim 57, wherein the gene is cloned in at least one of a viral vector and a non-viral vector.

59. The method of claim 58, wherein said viral vector comprises at least one of adenoviruses, adeno-associated viruses, helper dependent adenoviruses and retroviral vectors.

60. The method of claim 57, wherein said gene encoding clusterin is administered to the subject by at least one of intra-venous, intra-hypothalamic, intracerebroventricular and intrathecal injection.

61. A method comprising:
at least one of treating and preventing anorexia in a subject, comprising administering to the subject a therapeutically-effective amount of a regulator configured to downregulate clusterin expression.

62. The method of claim 61, wherein the anorexia comprises at least one of cancer-related anorexia, anorexia nervosa and false anorexia.

63. The method of claim 61, wherein the regulator comprises at least one of RNAs mediating RNA interference against clusterin gene expression, transcription inhibitors of clusterin, translation inhibitors of transcribed clusterin mRNA and inhibitors of clusterin localization.

64. The method of claim 63, wherein said RNAs mediating RNA interference comprises at least one an antisense RNA, interfering RNA, short hairpin RNA and small interfering RNA.

65. The method of claim 64, wherein said short hairpin RNA is complementary to a cDNA comprising a nucleotide sequence including SEQ ID NO: 1.


SEQUENCE LISTING (1 sequence)

SEQ ID NO: 1 - DNA, 59 bp, Artificial Sequence
  cDNA for shRNA targeting nucleotides 444-462 of murine clusterin sequence
catagaacttcatgcaggtatttcaagagaatacctgcatgaagttctattttttccaa